(19) United States
(12) Patent Application Publication
(10) Pub. No.: US 2021/0349098 A1
(43) Pub. Date: Nov. 11, 2021

(54) CANCER DIAGNOSIS AND MONITORING APPARATUS, SYSTEMS AND METHODS THEREOF

(71) Applicant: GINA-LIFE DIAGNOSTICS LTD., Haifa (IL)

(72) Inventors: Shlomit YEHUDAI-RESHEF, Habonim (IL); Roni MICHAELY, Herzelia (IL); Daniella YEHEZKELY-HAYON, Kibbutz Yifat (IL)

(21) Appl. No.: 17/379,331

(22) Filed: Jul. 19, 2021

Related U.S. Application Data
(63) Continuation of application No. PCT/IL2020/050085, filed on Jan. 22, 2020.
(60) Provisional application No. 62/795,891, filed on Jan. 23, 2019.

(51) Int. Cl. G01N 33/574; A61B 10/00; C12Q 1/6886; G01N 33/52; A61F 13/84; A61F 13/472; A61F 13/20

(52) U.S. Cl. CPC G01N 33/57449; A61B 10/0045; C12Q 1/6886; G01N 33/528; A61F 13/84; A61F 13/472; A61F 2013/47281; A61B 2010/0074; A61B 2010/0006; C12Q 2600/178; A61F 2013/8488; A61F 2013/8491; A61F 13/2077

(57) ABSTRACT

Disclosed herein a female hygienic device for diagnosing and/or monitoring cancer, comprising at least one absorption zone for accumulating vaginal discharge; and at least one indication zone comprising at least one agent for visually reacting with a physiological marker, the physiologic marker is indicative of a cancerous condition.

### RELATED APPLICATION

This application is a continuation of PCT/IL2020/050085 filed Jan. 22, 2020, which claims the benefit of priority under 35 USC § 119(e) of U.S. Provisional Patent Application No. 62/795,891 filed 23 Jan. 2019, the contents of which are incorporated herein by reference in their entirety.

### FIELD OF THE INVENTION

The present invention generally relates to diagnosis and/or monitoring systems, and more specifically, the present invention pertains to women's sanitary products embedded with cancer diagnostic and/or monitoring means.

### BACKGROUND OF THE INVENTION

Among the cancers that most often affect women are breast, colon, endometrial, lung, cervical, skin, and ovarian cancers. Ovarian Cancer is cancer of the ovaries, which are female reproductive organs that are similar to the testes in men. They produce the ova (eggs) that, when fertilized, develop into a fetus, and also generate the female sex hormones estrogen and progesterone. There are two ovaries, each of which is located within the pelvis beside the uterus.

When an ovary releases an egg, the egg follicle bursts open and becomes the corpus luteum. This structure needs to be repaired by dividing cells in the ovary. Continuous ovulation for a long time means more repair of the ovary by dividing cells, which might acquire mutations in each division, and may eventually lead to cancer.

Signs and symptoms of ovarian cancer are frequently absent in early stages, and even when they do exist, they may be subtle. This unfortunately leads in many cases to late diagnosis, having the cancer already spread and advanced into later stages. There is reason to believe that improved regular screening routines for ovarian cancer could lead to earlier diagnosis.

The risk of ovarian cancer increases in women who have ovulated more over their lifetime. This includes those who have never had children, those who begin ovulation at a younger age or reach menopause at an older age. Other risk factors include hormone therapy after menopause, fertility medication, and obesity. Ovarian cancer is more likely to occur as women get older. Women who have never had children, who have unexplained infertility, or who had their first child after age 30 may be at increased risk for this cancer. Women with a personal or family history of hereditary non-polyposis colon cancer (HNPCC), ovarian cancer, or breast cancer are more likely to have this disease.

About 10% of cases have been found to be related to inherited genetic risk; women with mutations in the genes BRCA1 or BRCA2 have about a 50% chance of developing the disease. Only one allele need be mutated to place a person at high risk, because the risky mutations are autosomal dominant.

If caught and treated in an early stage, ovarian cancer may be curable. Treatment usually includes some combination of surgery, radiation therapy, and chemotherapy. Outcomes depend on the extent of the disease and the subtype of the cancer present.

Several markers have been associated with women related cancer diagnosis and a few of which may be detected by diagnostic tests such as blood tests or feminine discharge extraction (for example in Van Gorp et al., “HE4 and CA125 as a diagnostic test in ovarian cancer: prospective validation of the Risk of Ovarian Malignancy Algorithm”, Br J Cancer. 2011; Leung et al., “Ovarian cancer biomarkers: current state and future implications from high-throughput technologies”, Adv Clin Chem. 2014; Di leva et al., “The Role of microRNAs in the Tumorigenesis of Ovarian Cancer”, Front Oncol. 2013).

Available tests are generally lab based and/or require clinical sample collection. For a woman to get tested she may need to actively turn to her physician and request a lab-based diagnostic test. Often a woman will request such a test only after having experienced at least some symptoms. Unfortunately, experiencing such symptoms often means it is already too late. For example, for women at risk, bi-yearly routine (serum CA-125) lab tests are often recommended. This test has not proven effective in early detection of ovarian cancer.

### SUMMARY OF THE INVENTION

Example 1: A female hygienic device for diagnosing and/or monitoring cancer, comprising: at least one absorption zone for accumulating vaginal discharge; and at least one indication zone comprising at least one agent for visually reacting with a physiological marker, the physiologic marker is indicative of a cancerous condition.

Example 2: The device of example 1, wherein the vaginal discharge comprises cervix mucus.

Example 3: The device of example 1, wherein the vaginal discharge comprises fallopian secretions.

Example 4: The device of any of examples 1-3, wherein the absorption zone is adapted to accumulate a predetermined quantity of the vaginal discharge.

Example 5: The device of any of examples 1-4, wherein the hygienic device further comprises at least one viscosity-reducing agent.

Example 6: The device of any of examples 1-5, wherein the indication zone comprises a lateral flow test strip and/or immunodetection based device and/or a solid phase immunodetection methodology. The immune-detection device may use a standard protein and/or multiple biomarkers.

Example 7: The device of example 6, wherein the lateral flow strip comprises at least two areas having the at least one agent.

Example 8: The device of example 7, wherein each of the at least two areas comprises a different amount of the at least one agent.

Example 9: The device of any of examples 1-8, wherein the indication zone further comprises at least one second agent for visually reacting with a second physiologic marker, the second physiologic marker is a not indicative of a cancerous condition.

Example 10: The device of any of examples 1-9, wherein the female hygienic device is a sanitary pad.

Example 11: The device of any of examples 1-10, wherein the female hygienic device is a tampon.

Example 12: The device of any of examples 1-11, wherein the female hygienic device further comprises a barrier film having at least one orifice, the at least one orifice aligned with the at least one absorption zone.

Example 13: The device of example 12, wherein the barrier film comprises a hydrophobic composition.

Example 14: The device of any of examples 1-13, wherein the at least one physiologic marker comprises CA125, HE4 or both.

Example 15: The device of any of examples 9-14, wherein the at least one second physiologic marker comprises at least one of the group consisting of albumin, actin, tubulin, a secondary antibody and any combination thereof.

Example 16: The device of any of examples 5-15, wherein the at least one viscosity-reducing agent comprises an agent for targeting the enzymatic activity of mucin.

Example 17: The device of any of examples 5-16, wherein the viscosity-reducing agent is selected from the group consisting of water beads, polyacrylamide, superabsorbent polymer, N-acetyl-L-cystein, N-acetylcystamine, Sodium thioglycollate, 2-mercaptoethanol, B-mercaptoethanol, viscosity reducing enzymes, acetic acid, ammonium acetate, (NH4)2SO4, perchloric acid, NaOH, DTT, Urea, SDS, Sodium Chloride and any combination thereof.

Example 18: The device of any of examples 1-17, wherein the agent comprises a DNA probe, and/or an RNA probe.

Example 19: The device of any of examples 1-18, wherein the agent comprises an antibody.

Example 20: The device of any of examples 1-19, wherein the at least one indication zone further comprises a detection enhancer.

Example 21: The device of example 19, wherein the detection enhancer is selected from the group consisting of gold nanoparticles, gold microparticles, polystyrene beads, cellulose nanobeads, fluorescent beads and any combination thereof

Example 22: The device of any of examples 1-21, wherein the visual reaction comprises a color change, and/or a color intensity change and/or fluorescent signal.

Example 23: The device of any of examples 1-22, wherein the female hygienic device further comprises a three-dimensional configuration sized and shaped for allowing the feminine discharge to drain to a direction of the at least one absorption zone.

Example 24: The device of any of examples 1-23, wherein the visual reaction comprises a marking indicative of a predetermined concentration of the at least one cancer biomarker.

Example 25: The device of any of examples 4-24, further comprising a visual indicator for indicating the predetermined quantity of the vaginal discharge.

Example 26: A kit for diagnosing and/or monitoring cancer in females, comprising the female hygienic device of any of examples 1-25, and a container comprising at least one viscosity-reducing agent.

Example 27: A system for diagnosing and/or monitoring cancer in females, comprising the female hygienic device of any of examples 1-26, and further comprising:

a sensor for detecting the visual reaction.

a processor configured for executing instructions for correlating the detected visual reaction with a predetermined quantity of the at least one physiologic marker.

Example 28: The system of example 27, wherein the sensor is a camera.

Example 29: The system of any of examples 27-28, wherein the processor is embedded in a camera (“reader”), mobile phone, a tablet, a personal computer, a server, a cloud-like server and any combination thereof.

Example 30: The system of any of examples 27-29, wherein the processor has instructions for notifying a medical personnel once the correlated quantity of the at least one physiologic marker exhibits an increase and/or decrease in quantity.

Example 31: A method for diagnosing and/or monitoring cancer with a female hygienic device, comprising:

absorbing in a hygienic device vaginal discharges of a user;

detecting at least one physiologic marker in the vaginal discharges, the physiologic marker level is indicative of a cancerous condition;

visually presenting the detection of the at least one physiological marker.

Example 32: The method of example 31, wherein the vaginal discharge comprises cervix mucus.

Example 33: The method of example 31, wherein the vaginal discharge comprises fallopian secretions.

Example 34: The method of any of examples 31-33, wherein the absorbing comprises accumulating a predetermined quantity of the vaginal discharge.

Example 35: The method of example 34, wherein the detecting is conducted after the accumulating of the predetermined quantity.

Example 36: The method of example 35, wherein the detecting is conducted by allowing a viscosity-reducing agent to contact the vaginal discharge.

Example 37: The method of any of examples 31-36, wherein the visually presenting comprises presenting a color or modifying an existing color and/or a fluorescent signal.

Example 38: The method of any of examples 31-37, wherein the visually presenting comprises presenting a symbol.

Example 39: The method of any of examples 37-38, comprising quantitatively assessing the visual presentation.

Example 40: The method of example 39, wherein the quantitative assessment comprises comparing the visual presentation to a predetermined concentration of the physiological marker.

Example 41: The method of example 39, wherein the quantitative assessment comprises comparing the visual presentation to a predetermined range of concentrations of the physiological marker.

Example 42: The method of any of examples 39-41, further comprising maintaining a log of the quantitative assessment, and comparing a recent quantitative assessment to previous assessments in the log.

Example 43: The method of example 42, comprising alerting a medical personnel when detecting an increase and/or decrease in a recent quantitative assessment as compared to previous assessments in the log.

Example 44: The method of any of examples 31-43, wherein the presentation of the detection of the at least one physiological marker is provided following a hygienic usage of the hygienic device, without performing additional modifications to the hygienic device by the user.

Example 45: The method of any of examples 31-43, wherein the presentation of the detection of the at least one physiological marker is provided following a hygienic usage of the hygienic device, followed by introducing a viscosity reducing agent to the hygienic device.

It is thus one object of the present invention to provide cancer diagnosis and monitoring system comprising: a carrier substrate adapted to at least partially contact a user's vagina; and a feminine discharge system incorporated into the carrier substrate, comprising at least one feminine discharge absorption zone, and at least one discharge indication zone comprising at least one cancer biomarker indicator; wherein the system further comprises a viscosity-reducing agent configured for contacting the absorption zone and for facilitating flow of reduced viscosity discharge to the at least one cancer biomarker indicator, the indicator visibly transformable post-contact with the at least one cancer biomarker; further wherein the feminine discharge is selected from the group consisting of vaginal discharge, uterine discharge, ovary discharge, fallopian discharge, cervix discharge and any combination thereof.

It is still an object of the present invention to provide the system as disclosed in any of the above, wherein the viscosity-reducing agent is provided in a manner selected from the group consisting of incorporated into the absorption zone, incorporated into the entire surface of the carrier substrate, provided separately from the carrier substrate and configured for applying onto the carrier substrate, and any combination thereof.

It is still an object of the present invention to provide the system as disclosed in any of the above, wherein the carrier substrate is selected from a group consisting of a sanitary pad, a tampon, an interlabial pad, a panty liner and any combination thereof.

It is still an object of the present invention to provide the system as disclosed in any of the above, wherein the carrier substrate further comprises a barrier film having at least one orifice, for isolating the feminine discharge system while enabling penetration of the feminine discharge into the feminine discharge system.

It is still an object of the present invention to provide the system as disclosed in any of the above, wherein the feminine discharge system is a lateral flow test strip.

It is still an object of the present invention to provide the system as disclosed in any of the above, wherein the at least one cancer biomarker is selected from the group comprising of CA125, HE4 and any combination thereof.

It is still an object of the present invention to provide the system as disclosed in any of the above, additionally comprising at least one second cancer biomarker selected from the group consisting of 22-1-1, leptin, prolactin, osteoponin, insulin-like growth factor II, macrophage inhibitory factor, CD44, soluble CD 54, miRNA 21, miRNA 92, miRNA 93 and any combination thereof.

It is still an object of the present invention to provide the system as disclosed in any of the above, further comprising at least one indicator for a non-cancerous biomarker.

It is still an object of the present invention to provide the system as disclosed in any of the above, wherein the non-cancerous biomarker is selected from the group comprising of albumin, actin, tubulin, a secondary antibody and any combination thereof.

It is still an object of the present invention to provide the system as disclosed in any of the above, wherein the viscosity-reducing agent is selected from the group consisting of water beads, polyacrylamide, superabsorbent polymer, N-acetyl-L-cystein, N-acetylcystamine, Sodium thioglycollate, 2-mercaptoethanol, B-mercaptoethanol, viscosity reducing enzymes, acetic acid, ammonium acetate, (NH4)2SO4, perchloric acid, NaOH, DTT, Urea, SDS, sodium chloride and any combination thereof.

It is still an object of the present invention to provide the system as disclosed in any of the above, wherein the indicator is selected from the group consisting of a DNA probe, an RNA probe, an antibody and any combination thereof.

It is still an object of the present invention to provide the system as disclosed in any of the above, wherein the indicator further comprises detection enhancing means selected from the group consisting of gold nanoparticles, gold microparticles, polystyrene beads, cellulose nanobeads and any combination thereof.

It is still an object of the present invention to provide the system as disclosed in any of the above, wherein the visible reaction is selected from the group consisting of a color change, a color intensity change and any combination thereof.

It is still an object of the present invention to provide the system as disclosed in any of the above, wherein the carrier substrate further comprises at least one region having a hydrophobic layer.

It is still an object of the present invention to provide the system as disclosed in any of the above, wherein the carrier substrate comprises at least one window for visual viewing of the at least one cancer biomarker indicator.

It is still an object of the present invention to provide the system as disclosed in any of the above, wherein the window is located at the posterior or anterior portion of the carrier substrate.

It is still an object of the present invention to provide the system as disclosed in any of the above, wherein the carrier substrate further comprises a three-dimensional configuration adapted for allowing the feminine discharge to drain to a direction of the feminine discharge system.

It is still an object of the present invention to provide the system as disclosed in any of the above, wherein the indicator's visible transformation is correlated with a predetermined concentration of the at least one cancer biomarker.

It is further an object of the present invention to provide a cancer diagnosis and monitoring apparatus comprising: a carrier substrate adapted to at least partially contact a user's vagina; and a feminine discharge system incorporated into the carrier substrate, comprising at least one feminine discharge absorption zone, and at least one discharge indication zone comprising at least one cancer biomarker indicator; wherein the absorption zone further comprises a viscosity-reducing agent for facilitating flow of the reduced viscosity discharge to the at least one cancer biomarker indicator, the indicator visibly transformable post-contact with the at least one cancer biomarker; further wherein the feminine discharge is selected from the group consisting of vaginal discharge, uterine discharge, ovary discharge, fallopian discharge, cervix discharge and any combination thereof.

It is another object of the present invention to provide a system for cancer diagnosis and monitoring comprising an apparatus, the apparatus comprising a carrier substrate adapted to at least partially contact a user's vagina, and a feminine discharge system incorporated into the carrier substrate, comprising at least one feminine discharge absorption zone, and at least one discharge indication zone comprising at least one cancer biomarker indicator; wherein the system further comprises a viscosity-reducing agent for dissolving the at least one feminine discharge to facilitate dissolved discharge into contact with the at least one cancer biomarker indicator, the indicator visibly transformable post-contact with the at least one cancer biomarker; further wherein the system further comprises a sensor for detecting the indicator's visible transformation and communicating such to a processor configured for executing instructions for correlating the indicator's visible transformation with a predetermined quantity of the at least one cancer biomarker; further wherein the feminine discharge is selected from the group consisting of vaginal discharge, uterine discharge, ovary discharge, fallopian discharge, cervix discharge and any combination thereof.

It is also an object of the present invention to provide the abovementioned system, wherein the sensor is a camera.

It is also an object of the present invention to provide the abovementioned system, wherein the processor is located in a device selected from the group consisting of a mobile phone, a tablet, a personal computer, a server, a cloud-like server and any combination thereof.

It is also an object of the present invention to provide the abovementioned system, wherein the processor is configured for notifying medical personnel once the correlated quantity of the at least one cancer biomarker exhibits an increase in quantity.

It is yet another object of the present invention to disclose a method of manufacturing a cancer diagnosis and monitoring apparatus comprising steps of: providing a carrier substrate adapted to at least partially contact a user's vagina; and incorporating a feminine discharge system into the carrier substrate, the feminine discharge system comprising at least one feminine discharge absorption zone, and at least one discharge indication zone comprising at least one cancer biomarker indicator; wherein the step (b) further comprises steps of incorporating a viscosity-reducing agent in the absorption zone for dissolving the at least one feminine discharge, thereby facilitating dissolved discharge into contact with the at least one cancer biomarker indicator, the indicator visibly transformable post-contact with the at least one cancer biomarker.

It is still an object of the present invention to provide the aforementioned method in any of the above, further comprising steps of selecting the carrier substrate from a group consisting of a sanitary pad, a tampon, an interlabial pad, a panty liner and any combination thereof.

It is still an object of the present invention to provide the aforementioned method in any of the above, further comprising steps of incorporating in the carrier substrate a barrier film having at least one orifice, for isolating the viscosity-reducing agent while enabling penetration of the feminine discharge into the feminine discharge system.

It is still an object of the present invention to provide the aforementioned method in any of the above, further comprising steps of incorporating the feminine discharge system in the form of a lateral flow test strip.

It is still an object of the present invention to provide the aforementioned method in any of the above, further comprising steps of selecting the at least one cancer biomarker from the group comprising of CA125, HE4 and any combination thereof.

It is still an object of the present invention to provide the aforementioned method in any of the above, further comprising steps of additionally incorporating in the feminine discharge system at least one second cancer biomarker and selecting such from the group consisting of 22-1-1, leptin, prolactin, osteoponin, inslin-like growth factor II, macrophage inhibitory factor, CD44, soluble CD 54, miRNA 21, miRNA 92, miRNA 93 and any combination thereof.

It is still an object of the present invention to provide the aforementioned method in any of the above, further comprising steps of incorporating in the feminine discharge system at least one indicator for a non-cancerous biomarker.

It is still an object of the present invention to provide the aforementioned method in any of the above, further comprising steps of selecting the non-cancerous biomarker from the group comprising of albumin, actin, tubulin, a secondary antibody and any combination thereof.

It is still an object of the present invention to provide the aforementioned method in any of the above, further comprising steps of selecting the viscosity-reducing agent from the group consisting of water beads, polyacrylamide, superabsorbent polymer, N-acetyl-L-cystein, N-acetylcystamine, Sodium thioglycollate, 2-mercaptoethanol, B-mercaptoethanol, viscosity reducing enzymes, acetic acid, ammonium acetate, (NH4)2SO4, perchloric acid, NaOH, DTT, Urea, SDS, Sodium Chloride and any combination thereof.

It is still an object of the present invention to provide the aforementioned method in any of the above, further comprising steps of selecting the indicator from the group consisting of a DNA probe, an RNA probe, an antibody and any combination thereof.

It is still an object of the present invention to provide the aforementioned method in any of the above, further comprising steps of incorporating in the indicator detection enhancing means selected from the group consisting of gold nanoparticles, gold microparticles, polystyrene beads, cellulose nanobeads and any combination thereof.

It is still an object of the present invention to provide the aforementioned method in any of the above, further comprising steps of adapting the visible reaction to be in a manner selected from the group consisting of a color change, a color intensity change and any combination thereof

It is still an object of the present invention to provide the aforementioned method in any of the above, further comprising steps of incorporating in the carrier substrate at least one region having a hydrophobic layer.

It is still an object of the present invention to provide the aforementioned method in any of the above, further comprising steps of incorporating in the carrier substrate at least one window for visual viewing of the at least one cancer biomarker indicator.

It is still an object of the present invention to provide the aforementioned method in any of the above, wherein the step of incorporating the window is provided by locating the window at the posterior portion of the carrier substrate.

It is still an object of the present invention to provide the aforementioned method in any of the above, further comprising steps of providing the carrier substrate in a three-dimensional configuration, thereby allowing the feminine discharge to drain to a direction of the feminine discharge system.

It is still an object of the present invention to provide the aforementioned method in any of the above, further comprising incorporating in the feminine discharge system a plurality of the cancer biomarker indicators and configuring each of the cancer biomarker indicators provides indication to a predetermined range of the biomarker concentration.

It is still an object of the present invention to provide the aforementioned method in any of the above, further comprising steps of correlating the indicator's visible transformation with a predetermined concentration range of the at least one cancer biomarker.

It is also an object of the present invention to provide a kit for cancer diagnosis and monitoring comprising: a cancer diagnosis and monitoring apparatus comprising: a carrier substrate adapted to at least partially contact a user's vagina; and a feminine discharge system incorporated into the carrier substrate, comprising at least one feminine discharge absorption zone, and at least one discharge indication zone comprising at least one cancer biomarker indicator; and a viscosity-reducing agent adapted for applying onto the absorption zone, for dissolving the at least one feminine discharge to facilitate dissolved discharge into contact with the at least one cancer biomarker indicator, the indicator visibly transformable post-contact with the at least one cancer biomarker; and an instructions manual.

It is still an object of the present invention to provide the abovementioned kit, wherein feminine discharge system comprises a plurality of the cancer biomarker indicators, and each of the cancer biomarker indicators provides indication to a predetermined range of the biomarker concentration.

It is still an object of the present invention to provide the abovementioned kit, further comprising a log manager for maintaining a biomarker quantity log including an entry for each detected biomarker.

It is yet another object of the present invention to provide a cancer self-monitoring system comprising: a carrier substrate adapted to at least partially contact a user's vagina; and a feminine discharge system incorporated into the carrier substrate, comprising at least one feminine discharge absorption zone, and at least one discharge indication zone comprising a plurality of cancer biomarker indicators; wherein the system further comprises a viscosity- reducing agent configured for contacting the absorption zone and for facilitating flow of reduced viscosity discharge to the plurality of cancer biomarker indicators, the indicators visibly transformable post-contact with the plurality of cancer biomarker; further wherein each of the cancer biomarker indicators provides indication to a predetermined range of the biomarker concentration; further wherein the feminine discharge is selected from the group consisting of vaginal discharge, uterine discharge, ovary discharge, fallopian discharge, cervix discharge and any combination thereof.

It is also an object of the present invention to provide the abovementioned system as disclosed in any of the above, wherein the viscosity-reducing agent is provided in a manner selected from the group consisting of incorporated into the absorption zone, incorporated into the entire surface of the carrier substrate, provided separately from the carrier substrate and configured for applying onto the carrier substrate, and any combination thereof.

It is also an object of the present invention to provide the abovementioned system as disclosed in any of the above, wherein the carrier substrate is selected from the group consisting of a sanitary pad, a tampon, an interlabial pad, a panty liner and any combination thereof.

It is also an object of the present invention to provide the abovementioned system as disclosed in any of the above, wherein the carrier substrate further comprises a barrier film having at least one orifice, for isolating the feminine discharge system while enabling penetration of the feminine discharge into the feminine discharge system.

It is also an object of the present invention to provide the abovementioned system as disclosed in any of the above, wherein the feminine discharge system is a lateral flow test strip.

It is also an object of the present invention to provide the abovementioned system as disclosed in any of the above, wherein the plurality of cancer biomarkers comprise at least one cancer biomarker selected from the group comprising of CA125, HE4 and any combination thereof

It is also an object of the present invention to provide the abovementioned system as disclosed in any of the above, wherein the plurality of cancer biomarkers comprise at least one cancer biomarker selected from the group consisting of 22-1-1, leptin, prolactin, osteoponin, inslin-like growth factor II, macrophage inhibitory factor, CD44, soluble CD 54, miRNA 21, miRNA 92, miRNA 93 and any combination thereof.

It is also an object of the present invention to provide the abovementioned system as disclosed in any of the above, further comprising at least one indicator for a non-cancerous biomarker.

It is also an object of the present invention to provide the abovementioned system as disclosed in any of the above, wherein the non-cancerous biomarker is selected from the group comprising of albumin, actin, tubulin, a secondary antibody and any combination thereof.

It is also an object of the present invention to provide the abovementioned system as disclosed in any of the above, wherein the viscosity-reducing agent is selected from the group consisting of water beads, polyacrylamide, superabsorbent polymer, N-acetyl-L-cystein, N-acetylcytamine, Sodium thioglycollate, 2-mercaptoethanol, B-mercaptoethanol, viscosity reducing enzymes, acetic acid, ammonium acetate, (NH4)2SO4, perchloric acid, NaOH, DTT, Urea, SDS, Sodium Chloride and any combination thereof.

It is also an object of the present invention to provide the abovementioned system as disclosed in any of the above, wherein the indicator is selected from the group consisting of a DNA probe, an RNA probe, an antibody and any combination thereof.

It is also an object of the present invention to provide the abovementioned system as disclosed in any of the above, wherein the indicator further comprises detection enhancing means selected from the group consisting of gold nanoparticles, gold microparticles, polystyrene beads, cellulose nanobeads and any combination thereof.

It is also an object of the present invention to provide the abovementioned system as disclosed in any of the above, wherein the visible reaction is selected from the group consisting of a color change, a color intensity change and any combination thereof.

It is also an object of the present invention to provide the abovementioned system as disclosed in any of the above, wherein the carrier substrate further comprises at least one region having a hydrophobic layer.

It is also an object of the present invention to provide the abovementioned system as disclosed in any of the above, wherein the carrier substrate comprises at least one window for visual viewing of the at least one cancer biomarker indicator.

It is also an object of the present invention to provide the abovementioned system as disclosed in any of the above, wherein the window is located at the posterior or anterior portion of the carrier substrate.

It is also an object of the present invention to provide the abovementioned system as disclosed in any of the above, wherein the carrier substrate further comprises a three-dimensional configuration adapted for allowing the feminine discharge to drain to a direction of the feminine discharge system.

It is also an object of the present invention to provide the abovementioned system as disclosed in any of the above, wherein the indicator's visible transformation is correlated with the predetermined concentration of the plurality of cancer biomarkers.

It is also an object of the present invention to provide the abovementioned system as disclosed in any of the above, further comprising a sensor adapted for detecting the indicator's visible transformation and communicating such to a processor configured for executing instructions for correlating the indicator's visible transformation with a predetermined quantity of the at least one cancer biomarker.

It is also an object of the present invention to provide the abovementioned system as disclosed in any of the above, wherein the sensor is a camera.

It is also an object of the present invention to provide the abovementioned system as disclosed in any of the above, wherein the processor is located in a device selected from the group consisting of a mobile phone, a tablet, a personal computer, a server, a cloud-like server and any combination thereof.

It is lastly an object of the present invention to provide the abovementioned system as disclosed in any of the above, wherein the processor is configured for notifying medical personnel once the correlated quantity of the at least one cancer biomarker exhibits an increase in quantity.

### BRIEF DESCRIPTION OF THE DRAWINGS

The novel features believed to be characteristics of the invention are set forth in the appended examples. The invention itself, however, as well as the preferred mode of use, further objects and advantages thereof, will best be understood by reference to the following detailed description of illustrative embodiment when read in conjunction with the accompanying drawings, wherein:

FIGS. 1a-c schematically presents the cancer diagnosis apparatus in accordance with an embodiment of the present invention, provided as a sanitary pad with a single absorption zone;

FIGS. 2a-c schematically present another embodiment of the present invention, provided as a sanitary pad with multiple absorption zones;

FIGS. 3a-b schematically present an embodiment of the present invention, provided as a sanitary pad comprising water repellent regions;

FIGS. 4a-c schematically present various embodiments illustrating a variety of indicators;

FIG. 5 schematically presents an embodiment of the present invention, provided as a tampon;

FIGS. 6a-b schematically present another embodiment of the present invention as provided in a sanitary pad, characterized by a three-dimensional structure for optimizing absorption of feminine discharges;

FIG. 7 schematically presents an explosive view of an embodiment of the present invention, illustrating a possible arrangement of the various layers which may be incorporated into the form of a sanitary pad;

FIG. 8 schematically illustrates use of an embodiment of the present invention in the form of a kit, comprising of the sanitary pad provided with externally applied viscosity reducing agent;

FIGS. 9a and b schematically illustrates an embodiment of the present invention providing a plurality of biomarker indicators, each having a predetermined range of marker concentration, wherein FIG. 9a provides an example of a diagnostic result and FIG. 9b provides an example of a change in the diagnostic result; and

FIGS. 10a and b schematically illustrate another embodiment of two diagnostic results, pertaining to a plurality of biomarker indicators indicated for at least two different biomarkers.

FIG. 11a demonstrates a quantification of total protein in vaginal fluids as compared to serum (i.e. blood sample). FIG. 11b shows detected quantities of CA-125 in the vaginal fluid as compared to the serum (averaged over samples taken from 4 healthy women), demonstrating the higher levels of CA-125 in vaginal fluids.

FIG. 12 also illustrates the higher concentration of CA-125 in vaginal fluids when comparedto other sampled fluids, such as saliva or blood.

FIG. 13 shows detection of CA-125 in a single patient, showing its concentration over time in the vaginal fluid and in the serum, taken over a period of chemotherapy treatment followed by remission.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the examples without some or all of these specific details. For the purpose of clarity, technical material that is known in the technical fields related to the invention has not been described in detail so that the present invention is not unnecessarily obscured.

Women may be sensitive and/or resistant to clinical based gathering of vaginal secretion, included and/or especially during a non-menstrual phase. In some embodiments, the current invention facilitates easier clinic based and/or home-based gathering and/or testing of vaginal secretions. For example, the system may include an anatomical pad(s) for gathering vaginal secretions and/or a biomarker reader adapted for the pad. Optionally the system is configured for continuous use and/or home use. Management of the pad and/or testing may be adaptive, for example, based on baseline(s) adapted on personalized data

An aspect of several embodiments of the current invention relates to diagnosing and/or monitoring cancer in females based on indications of a physiologic marker in a female hygienic product. As used herein, the term “hygienic product” refers to any sanitary apparatus used in female hygiene routines, such as for a non-limiting example, sanitary pads, and/or tampons, and/or interlabial pads, and/or pantyliners, and/or sanitary napkins, and/or sanitary towel, and/or menstrual pad. The means and methods provided by some embodiments of the present invention utilize feminine discharge (also referred to as vaginal discharge) for an identification of at least one physiologic marker suggestive of a female-related cancerous condition. In some embodiments, vaginal discharges include cervical mucus. Alternatively or additionally, the vaginal discharges include vaginal fluid comprising fallopian secretions. In some embodiments, a female-related cancerous condition includes fallopian cancer. Alternatively or additionally, the cancerous condition includes ovarian cancer, and/or uterine cancer, and/or cervical cancer and/or breast cancer. In some embodiments, the physiologic marker is a non-cancerous indicator, used for example, as a positive control of the device detection system. As used herein, vaginal discharge does not include menstrual fluids.

In some embodiments, the current invention provides women with a self-operable solution, which is simple and easy to follow on a regular basis. This may facilitate more consistent screening than conventional solutions

In some embodiments, the current invention provides a self-diagnostic device easy to operate at the comfort of one's home and/or directed for early diagnosis of women-related cancerous conditions fulfills a long-felt need.

An advantage of using vaginal discharge which is not menstrual fluids for the detection of the physiologic marker is the ability to identify the physiologic marker at least once a day. In some embodiments, the physiologic marker is identified every few days, for example a week. In other embodiments the physiologic marker is identified at least once a month, but not on menstruation days when the vaginal fluids are diluted in the menstruation fluids.

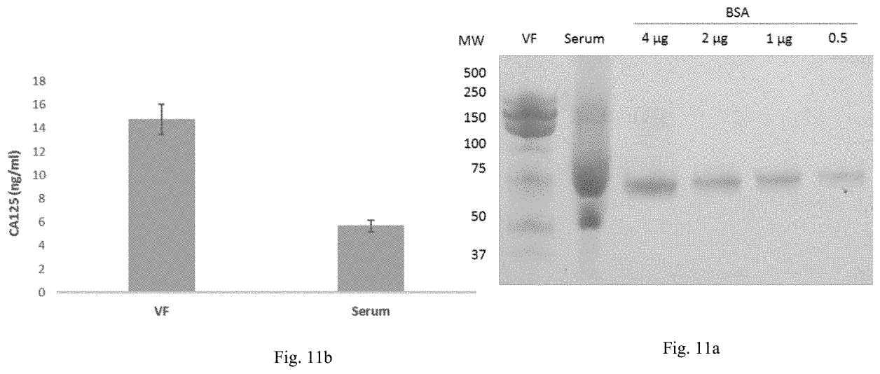

Therefore, another advantage of using vaginal discharge which is not menstrual fluids is the surprisingly high concentration of the physiological markers, which are not diluted in the menstruation fluids, as shown in FIG. 11. FIG. 11a demonstrates a quantification of total protein in vaginal fluids as compared to serum (i.e. blood sample). FIG. 11b shows detected quantities of CA-125 in the vaginal fluid as compared to the serum (averaged over samples taken from 4 healthy women), demonstrating the higher levels of CA-125 in vaginal fluids.

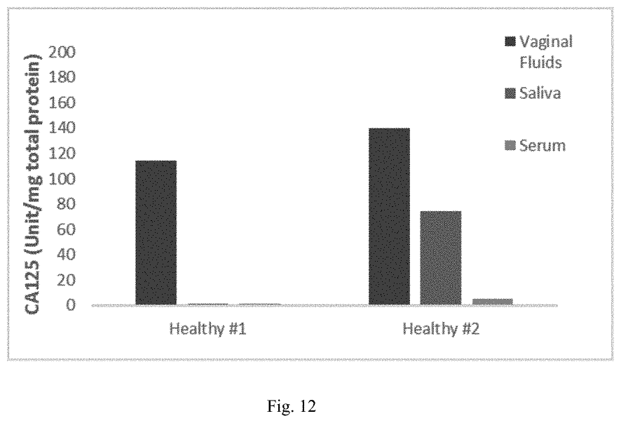

FIG. 12 also illustrates the higher concentration of CA-125 in vaginal fluids when compared to other sampled fluids, such as saliva or blood. It also demonstrates the variability between two individuals.

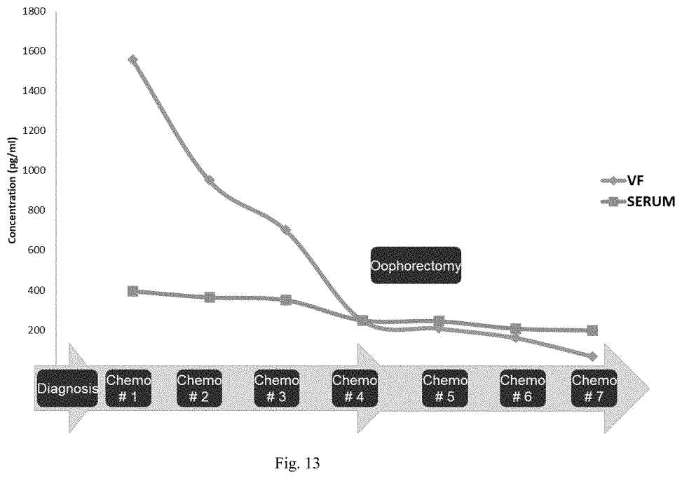

FIG. 13 shows detection of CA-125 in a single patient, showing its concentration over time in the vaginal fluid and in the serum, taken over a period of chemotherapy treatment followed by remission. The graph marked with diamond shape is CA-125 detected in the vaginal fluid and the graph marked with squares is the same marker detected in the serum. The graphs show that the sensitivity of detection in the vaginal fluid is higher than detection in the blood, even during the remission period, where the marker detection drops below the detected baseline in the serum.

In some embodiments, the hygienic device includes at least one absorption zone configured to accumulate vaginal discharges. In some embodiments, the absorption zone comprises a predetermined capacity for fluid accumulation. Optionally, a visual indicator is provided to indicate a full capacity, for example, a color change and/or an appearance or disappearance of a visual marker.

In some embodiments, vaginal discharges absorbed in the hygienic device are utilized to identify at least one physiologic marker related to a cancerous condition. For example, a physiologic marker may include CA125 and/or HE4. Other examples include 22-1-1, and/or leptin, and/or prolactin, and/or osteoponin, and/or inslin-like growth factor II, and/or macrophage inhibitory factor, and/or CD44, and/or soluble CD 54, and/or miRNA 21, and/or miRNA 92, and/or miRNA 93.

Yet other examples include Mesothelin, and/or M-CSF, and/or Osteopontin (OPN), and/or Expression of kallikrein (KLK)-related peptidases, and/or Preoperative high plasma bikunin level, and/or Plasma cell-free DNA, and/or VEGFs, and/or EphA2 expression, and/or FGF-1, and/or EZH2, and/or Claudin family members, and/or EGFR and/or HER2, and/or p53 mutation, and/or Cyclin D1, and/or cyclin E, and/or Serum sFas levels, and/or ERCC1, and/or IL-6, and/or IL-7, and/or IL-8, and/or Ascitic-fluid IL-12 levels, and/or APM (antigen processing machinery) component, and/or B7-H3, and/or B7-H4, and/or Intratumoral CD3+, and/or CD8+T cells infiltration, and/or Gamma-interferon expression, and/or Claudin-3, and/or Claudin-4, and/or MMP-2, and/or MMP-9, and/or MT1-MMP, and/or FAK, and/or Levels of miR-200, miR-141,miR-18a, miR-93, miR-429, let-7b, miR-199a, and/or Dicer, and/or Drosha expression.

Optionally, a viscosity-reducing agent is utilized with the device to allow fluidizing of the vaginal discharges, in order to facilitate their utilization in the diagnostic system of the diagnosis device. As used herein, the term “viscosity-reducing agent” refers to any solvent, buffer or gel-like beads which in contact with a viscous fluid interact with it to provide a more liquefied formulation of the viscous fluid, resulting in fluid with reduced viscosity. In some embodiments, the viscosity reducing agent is embedded with the hygienic device prior to its hygienic use, for example, by a manufacturer of the hygienic device. Alternatively or additionally, a viscosity reducing agent is added to the hygienic device by the user, optionally following its hygienic usage. In some embodiments, the viscosity-reducing agent is made in contact with the discharges only after the discharges are sufficiently accumulated, and/or after a predetermined quantity of the discharges has been accumulated. In some embodiments, the viscosity reducing agent is a mucin dissolving agent. Alternatively or additionally, the viscosity reducing agent is a chemical composition. Alternatively or additionally, viscosity reducing enzymes are used. As used herein, the term “enzymatic activity” refers to a catalytic effect exerted by an enzyme, which could be in the form of a protein having enzymatic activity capacity and/or any other material contributing to the catalytic effect in a chemical reaction, and such aiding cofactors (for example magnesium, adenosine triphosphate and the like).

In some embodiments, the diagnostic device provides diagnostic results only by using the device at its main hygienic usage, and without adding additional functions by the user. Alternatively, the user needs to perform at least one more function following the hygienic usage of the device. For example, a user may wear a hygienic device for several hours, after which the device is removed, and a dissolving agent is added to the device.

An aspect of several embodiments of the invention relates to quantitative and/or relative assessment of a presence of a physiologic marker in female discharges. In some embodiments, a female hygienic device is configured to absorb vaginal discharges, identify at least one physiologic marker and accordingly visually indicate the presence of the physiologic marker. In some embodiments, each user establishes a baseline of the presence of the physiologic marker, for example by using the device for the first time, or by averaging a number of device usages. In some embodiments, an image analysis method is used to identify variations from the user's baseline. Optionally, a smartphone is used, having a processor with instructions for analyzing the variations. Alternatively or additionally, a tablet, and/or a personal computer are used. In some embodiments, the smartphone, or the like, alerts a caregiver once a variation is determined. Optionally, a threshold of the variation is predetermined for inducing an alert.

In some embodiments, quantitative assessment is provided by assessing the color intensity of an indicator. Alternatively or additionally, quantitative assessment is provided by assessing the number of indicators, for example, between 1 and 3 indicators, or between 1 and 5 indicators, or between 1 and 10 indicators. Alternatively or additionally, quantitative assessment is provided by assessing the shape of the indicator/s.

An aspect of some embodiments of the present invention enables high-risk women to specifically, and personally follow their own changes and/or progressions in their physiologic conditions, by means of a platform for at-home monitoring. In some embodiments, the system enables high-risk women to establish a personal baseline, which optionally serves as a reference value. In some embodiments, women periodically, for example once a month, test their current values and compare them to their reference values. Any change in biomarker concentration indicated by a change from the reference values would inform the woman she needs to proceed to a thorough check-up to find out the source of this rise.

In some embodiments, the hygienic diagnostic device is used for following up on post oophorectomy patients, optionally, as routine screening. It should be noted that post oophorectomy patients are extremely liable to develop relapse and, in some embodiments, the device is used with a high frequency, such as every month.

In some embodiments, the current invention relates to a semi quantitative detection unit for multi biomarkers and/or for a biomarker fingerprint/signature. Optionally, the biodetection device may allow quantification of the conditions for example employing a standard protein. Optionally, the detection unit may include a lateral flow device for sampling and/or testing. Optionally, detection may include immunodetection.

In some embodiments, a bio-detection device may use another immune-detection method. For example, the bio-detection device may use a standard protein and/or multiple biomarkers. For example, the bio-detection device may include a solid phase immune-detection method for example bead based.

In some embodiments, an immune detection assay may be used for biomarker quantitation. Optionally, one or more assays may be antibody-based for example using enzymatic, fluorescent and/or nano-particles platforms. Such platforms may include one or more of bead based assay, lateral flow based assays, and/or solid phase immunoassays (e.g. such as ELISA).

In some embodiments, a testing platform may include buffer. For example, a buffer may be used to control pH and/or concentrations of salts (e.g. electro-conductivity) of a sample. Optionally, a buffer may contain protease and/or Nucleic acid digesting enzyme (e.g. DNase, RNase) inhibitors. Optionally, a buffer may contain a stabilizing protein and/or a carrier protein. Optionally a buffer may contain a standard protein (for example facilitating quantitation). In some embodiments, a buffer may contain a detection antibody. In some embodiments, a buffer may contain one or more pairs of antibodies and/or beads according to the number and kind of analytes, for example to measure biomarker concentrations. Optionally a buffer may contain an antibody pair conjugated to detect a moiety (e.g. fluorescent/gold nano particles/enzymes). Optionally a buffer may contain primers for DNA/RNA detection.

In some embodiments, a vaginal fluid sampling and/or testing system may be configured to give consistent results under varying conditions. For example, the system may account for personal variations between subjects and/or temporal variations and/or variations in the state of a subject (e.g. breast feeding, mood, phase of menstruation cycle). For example, a sampling and/or testing method may include calculating the change between baseline and a specific measurement and, determining if this change is indicative for cancer presence. For example, an algorithm may be used predict the risk for ovarian cancer. The algorithm may use multiple test data and/or data measured over time and/or data about the individual and/or her state and/or data between different individuals. Optionally the algorithm will run on a personal computer of the user (e.g. a cell phone and/or a personal computer). Optionally the algorithm will use artificial intelligence. It may employ captured images of sampling and/or testing devices (e.g. the subject takes a picture and/or the algorithm interprets the picture). Alternatively or additionally, data storage and/or processing may be clinic based and/or network based. In some embodiments an algorithm will include data from personal data devices and/or network based data (e.g. cell phones, fitness equipment, gym records, smart watches, location services, weather services) to improve testing interpretation. An application on a personal data device (e.g. a cell phone) may collect and/or interpret and/or transmit data.

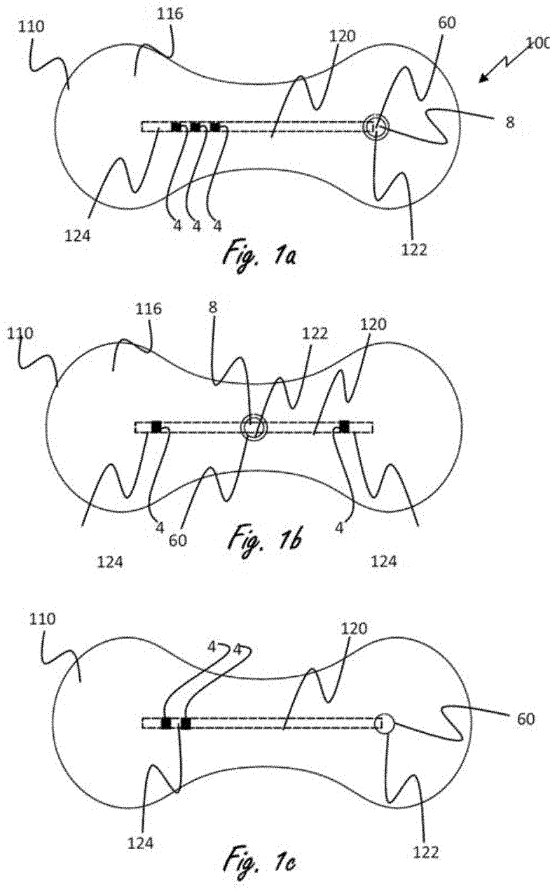

Reference is now made to FIG. 1 schematically presenting a female cancer diagnosis hygienic device 100, in accordance with some embodiments of the present disclosure. In some embodiments, diagnosis device 100 comprises a sanitary pad 110. In some embodiments, sanitary pad 110 includes a discharge diagnosis system 120, for example, a lateral flow test strip. Optionally, discharge diagnosis system 120 comprises at least one absorption zone 122 configured for accumulating vaginal discharges. In some embodiments, discharge diagnosis system comprises at least one indication zone 124, configured for identifying a presence of at least one physiologic marker in the vaginal discharges. FIGS. 1a and 1b illustrate various optional positions of absorption zone 122 and indication zone 124 along discharge diagnosis system 120.

In some embodiments, absorption zone 124 comprises an embedded viscosity-reducing agent 8, as illustrated in FIGS. 1a and 1b. Alternatively or additionally, the viscosity-reducing agent is provided distinctly from the sanitary pad 110, and is actively added to the discharge diagnosis system by applying it directly, optionally onto absorption zone 122. A potential advantage of including a viscosity-reducing agent is the liquefying of the feminine discharges (which are typically highly viscous), thereby facilitating their utilizations in the diagnostic system 120. For example, in some embodiments, when diagnostic system 120 is a lateral flow test strip, liquefying the viscous feminine discharge potentially enables its flow along the test strip all the way to the indication zone 124.

In some embodiments, a barrier 116 covers the outer surface of diagnostic device 110. A potential advantage of barrier 116 is to isolate the inner layers of the diagnostic device from the vaginal area of the user. Optionally, barrier 116 comprising at least one orifice 60 to allow for a penetration of feminine discharges into the inner layers of diagnostic device 110, including discharge diagnostic system 120. For example, barrier 116 may include non-woven cotton, and/or polyester, and/or polyethylene, and/or polypropylene, and/or nylon and/or rayon and/or formed thermoplastic films, which may or may not allow penetration of the feminine discharges. Optionally, the barrier comprises a material that is non-irritating to the contacting areas of the user.

In some embodiments, orifices for allowing discharge penetration, such as 60, further include absorption zones 122. Optionally, absorption zone comprises highly absorbent materials as well as viscosity-reducing agent 8. In some embodiments, highly absorbent materials are used and include, for example, any suitable hydrophilic fiber material such as cellulose, and/or modified cellulose, and/or rayon, and/or polyesters such as polyethylene terephtalate (DACRON [trademark]), and/or hydrophilic nylon (HYDROFIL [trademark]), and the like. FIGS. 1a and b illustrate various configurations of possible orifice 60 locations, and according to it, indication zone 124 locations.

In some embodiments, indication zone 124 comprises at least one cancer biomarker indicator, and optionally further comprises at least one non-cancerous biomarker, for example to serve as a control for the reliability of the performed test. In some embodiments, indication zone 124 comprises biomarker detectors for at least CA125 and HE4. Alternatively or additionally, indication zone 124 comprises additional biomarker detectors for 22-1-1, leptin, prolactin, osteoponin, inslin-like growth factor II, macrophage inhibitory factor, CD44, soluble CD 54, miRNA 21, miRNA 92 or miRNA 93.

In some embodiments, biomarker detectors are in the form of a DNA and/or RNA probes, and/or protein directed antibodies and/or derivatives thereof. Optionally, specificity enhancing formulations are provided, such as for example the addition of gold nanoparticle and/or microparticles, and/or polystyrene beads, and/or cellulose nanobeads and the like.

FIG. 1c illustrates the hygienic device 100 in accordance with some embodiments of the present invention having orifice 60 aligned with absorption zone 122, having no built-in viscosity-reducing agent. In some embodiments, the viscosity-reducing agent is included in a separate container provided as a kit with device 100, and adapted for usage by the user, for example by including a dripper. In some embodiments, after the hygienic usage of device 100, having vaginal discharges passed through orifice 60 and been absorbed in absorption zone 122, in this embodiment the user introduces the viscosity-reducing agent onto the absorption zone 122, initiating the liquefying of the discharge and facilitating their flow towards indication zones 124.

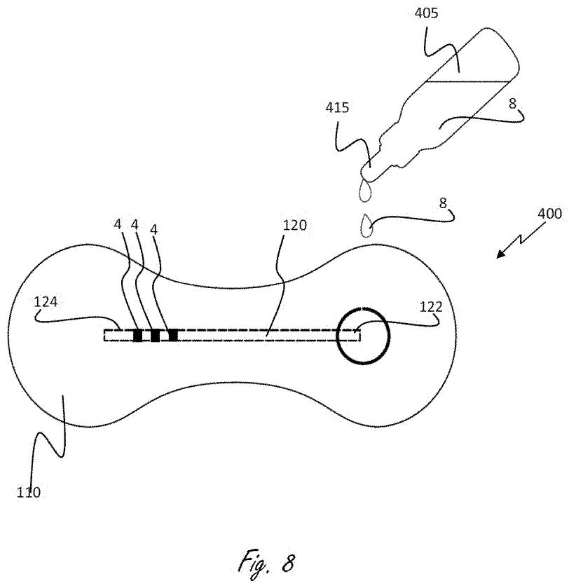

Alternatively or additionally, viscosity-reducing agent 8 is in an embedded form, which in a non-limiting example may be made of water beads, polyacrylamide, superabsorbent polymer, N-acetyl-L-cystein, N-acetylcystamine, Sodium thioglycollate, 2-mercaptoethanol, B-mercaptoethanol, viscosity reducing enzymes (mainly protease enzymes, such as in a non-limiting example trypsin), acetic acid, ammonium acetate, (NH4)2SO4, perchloric acid, NaOH, DTT, Urea, SDS, Sodium Chloride, any high concentration salt solution or the like, or it may be provided separately as part of a kit (see FIG. 8), and may incorporate the above exemplified materials contained in a container.

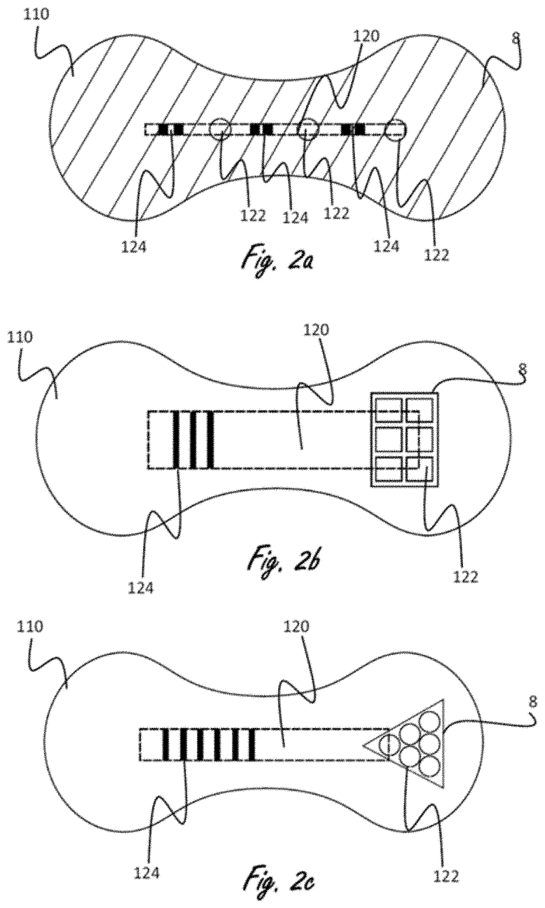

Reference is now made to FIG. 2, illustrating in a schematic manner a hygienic device 110 having feminine discharge system 120 in the form of a lateral flow test strip, having a plurality of absorption zones 122, in accordance with some embodiments of the invention. In some embodiments, absorption zones 122 are adapted for accumulating the vaginal discharges of a user. Optionally, this is achieved by incorporating highly absorbing materials into such vaginal collection zones 122. In some embodiments, an access to the absorption zone 122 is provided through orifices 60 in a barrier 116 covering the surface area of hygienic device 110. In some embodiments, absorption zones 122 are characterized by a variety of shapes, and/or sizes and/or arrangement, as depicted in FIGS. 2a-c.

According to some embodiments of the present invention, FIG. 2a illustrates three absorption zones characterized by round orifices 60 of barrier 116. In this embodiment, exemplified are three indication zones 124 are also available, each having two indicators 4. In some embodiments, viscosity-reducing agent 8 is introduced to the hygienic device 100 either above or beneath barrier 116.

In accordance with some embodiments of the present invention, FIG. 2b illustrates an altered arrangement of the plurality of absorption zones 122, having a substantially rectangular outline. For the manner of illustration, only six such absorption zones 122 are illustrated, however it should be noted that any number and/or any size of such zones may be provided. In some embodiments, viscosity-reducing agent 8 is provided as a region which encompasses the plurality of absorption zones 122, for example a rectangular region. In this embodiment, one portion of the vaginal discharge system 120 is in contact with absorption zones 122, while the other portion comprises the indication zone 124, which in this non-limiting example comprises three indicators 4. In some embodiments, indicators 4 comprise indicators for two cancer biomarkers, such as CA125 and HE4, and one non-cancerous biomarker. In some embodiments, a non-cancerous biomarker comprises any housekeeping gene such as for example albumin, actin or tubulin, and/or could be a secondary antibody, i.e. an antibody configured to bind another antibody, which may be derived, in a non-limiting example, from a mouse, rabbit, donkey, goat and the like.

In accordance with some embodiments of the present invention, illustrated in FIG. 2c is another suggested arrangement of the plurality of absorption zones 122, arranged in a triangular-like shape, optionally having its vertex directed towards vaginal discharge system 120. Exemplified is viscosity-reducing agent 8 encompassing the triangular area of the absorption zones 122.

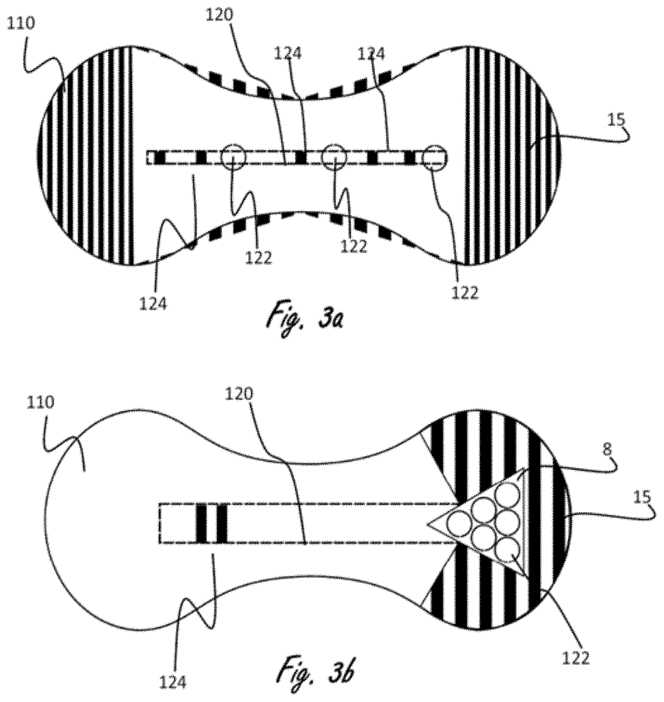

Reference is now made to FIG. 3, schematically illustrating the sanitary pad, as illustrated in FIG. 2, which is additionally incorporated with a hydrophobic, water repellent layer 15, for eliminating spillover of the feminine discharges into undesired regions, in accordance with some embodiments of the invention. In some embodiments, layer 15 is provided in the outskirts of the device, as illustrated in FIG. 3a. Alternatively or additionally, it surrounds the absorption zone 122 as illustrated in FIG. 3b.

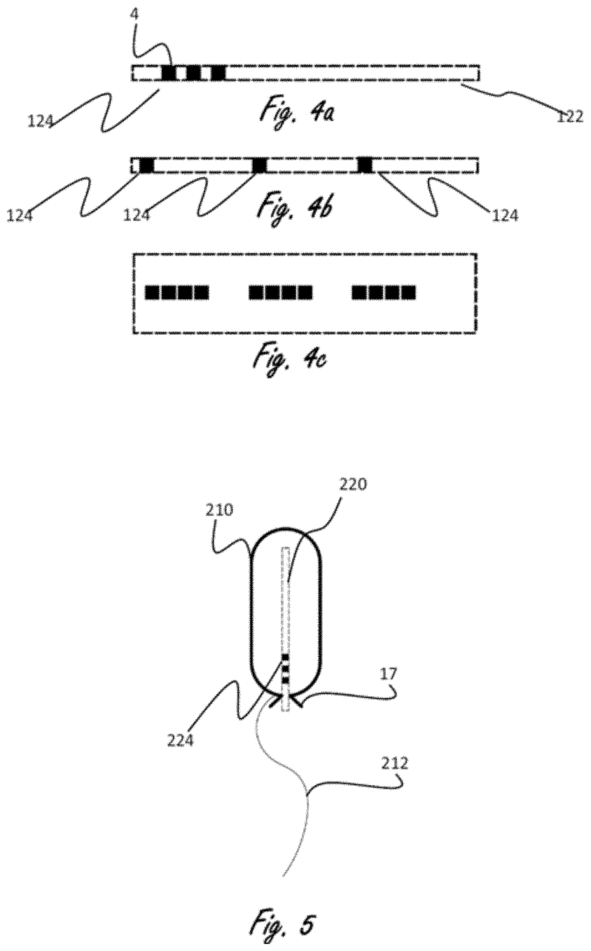

Reference is now made to FIG. 4, schematically illustrating the feminine discharge system 120 having various configurations of indication zones 124, in accordance with some embodiments of the invention. In some embodiments, the feminine discharge system is a lateral flow test strip, designed for allowing flow of feminine discharge and thus facilitating its contact with indication zones 124. FIG. 4a exemplifies having a single indication zone 124, comprising a plurality of biomarker indicators 4, in accordance with some embodiments of the invention. FIG. 4b exemplifies having a plurality of indication zones 124, each having at least one biomarker indicators 4, in accordance with some embodiments of the invention. FIG. 4c exemplifies having at least one indicator adapted to provide quantitative or semi-quantitative indications, in accordance with some embodiments of the invention. For example, each such indicator 4 comprises sub-indicators, each having a variable predetermined quantity of indicators, optionally in the form of antibodies, and/or nucleic acid probes and/or by means of enzymatic activity.

Reference is now made to FIG. 5, illustrating a tampon 210 is embedded with vaginal discharge system 120, in accordance with some embodiments of the invention. In some embodiments, tampon 210 is adapted to absorb vaginal discharges from a user, and it may or may not be additionally incorporated with a viscosity-reducing agent. In some embodiments, vaginal discharges are collected onto vaginal discharge system 120 and then flow to indication zone 124, having at least one biomarker indicator. In the exemplified embodiment of FIG. 5, results are obtained by removing the vaginal discharge system 120 by pulling its edge. Optionally, resecting lips 17 are provided for removing any excess fluids from the vaginal discharge system 120.

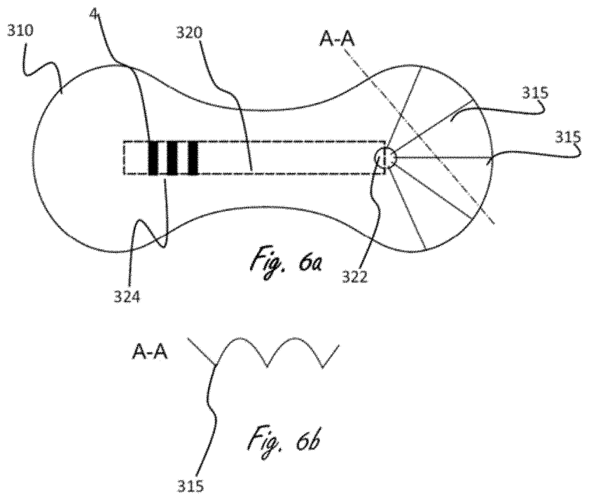

Reference is now made to FIG. 6, illustrating a sanitary pad having a three-dimensional configuration which potentially allows feminine discharges to flow and accumulate at the absorption zone 322, in accordance with some embodiments of the invention. In some embodiments, the three-dimensional configuration is in the form of recesses 315 which are shown in FIG. 6a in a top view, and in FIG. 6b as a cross-section view taken in line A-A shown in FIG. 6a. In some embodiments, the recesses are located over the carrier substrate 310, and are sized and shaped to structurally lead to the absorption zone 322 found in the embedded vaginal discharge system 320. Optionally, absorption zone 322 comprises viscosity-reducing agent or it can be applied externally after removing the sanitary pad for inspection by an externally provided container, which is in some embodiments provided as part of kit with the hygienic device. Once the vaginal discharges have reduced viscosity, their flow is facilitated along vaginal discharge system 320 and into contact with indication zone 324, having at least one biomarker indicator 4.

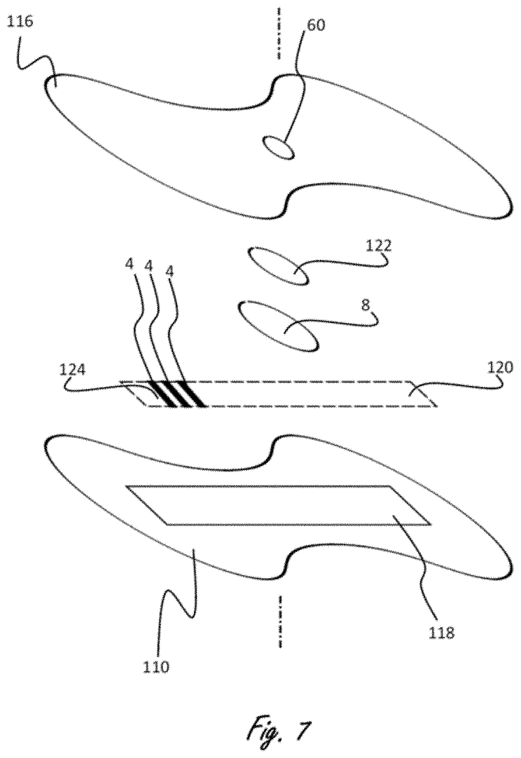

Reference is now made to FIG. 7, schematically illustrating an explosive three-dimensional view of a sanitary pad, and exposing variable layer which may be incorporated into this device, in accordance with some embodiments of the invention. In some embodiments, carrier substrate 110 is provided for mechanically supporting additional layers. In some embodiments, carrier substrate 110 comprises a window 118, enabling the visual inspection of at least part of the inner layers, and specifically, enabling the view of the indication zone 124, containing the indicators 4. Window 118 is provided in this non-limiting example at the posterior portion of the apparatus, but it should be noted that window 118 may also be provided at the anterior portion of the apparatus, and optionally, may be incorporated with a transparent protective layer. In some embodiments, indication zone 124 is embedded in a portion of vaginal discharge system 120 which is adapted to receive vaginal discharges from absorption zone 122. In some embodiments, indication zone 124 is visible from both directions, i.e. from a top view and a bottom view. Alternatively, it is viewed from a bottom view of the device. In some embodiments, encompassing the structure, is barrier 116, which isolates the various layers from the user's body, while still allowing penetration of the feminine discharges into the absorption zone through the at least one orifice 60.

Reference is now made to FIG. 8, illustrating in a schematic manner kit 400 in accordance with some embodiments of the invention. In some embodiments, kit 400 comprises a hygienic device 110, embedded with vaginal discharge system 120 containing an absorption portion 122 and detection zone 124, and including at least one detector 4. In some embodiments, kit 400 includes a container 405, for containing viscosity-reducing agent 8. In some embodiments, container 405 comprises a dripping portion 415 or a dropper or the like, for example for dripping a few drops of the viscosity-reducing agent 8 directly onto the absorption zone 122, where vaginal discharges are expected to be accumulated. Potentially, the dripping of viscosity-reducing agent 8 leads to the reduction in viscosity, or increased dissolvability or fluidity, of the vaginal discharges and accordingly onsets the flow of the dissolved vaginal discharges along the vaginal discharge system 120 and up to detection zone 124.

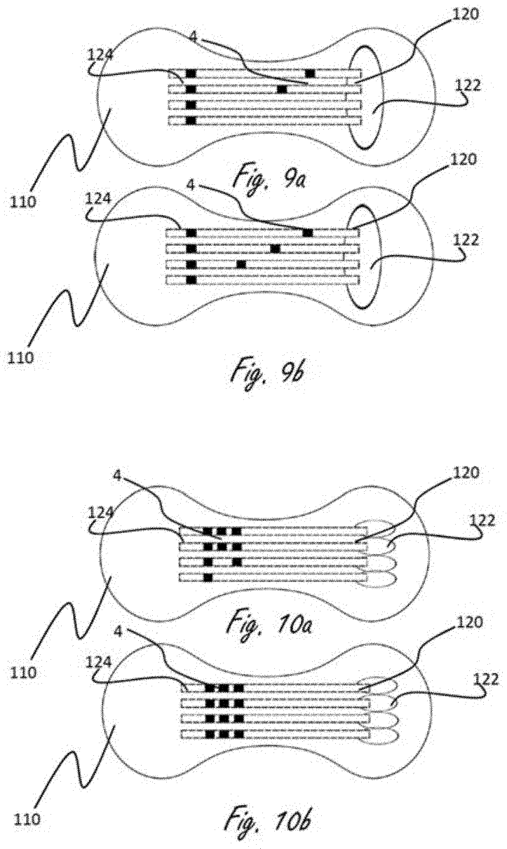

Reference is now made to FIG. 9, schematically illustrating a personal monitoring system in accordance with several embodiments of the invention. In some embodiments, the platform is provided in the form of a system, having a substrate carrier 110, being a disposable, optionally single-use, absorbent device such as a sanitary pad, a tampon, an interlabial pad, or a panty liner. In some embodiments, incorporated into substrate carrier 110 is a vaginal discharge system 120, or a plurality of such systems, provided with a discharge absorption zone 122 and indication zone 124. In some embodiments, the at least one discharge system 120 comprises in its indication zones a plurality of biomarker detectors 4, each is directed towards a different predetermined range of biomarker concentration (for a non-limiting exemplification see example 1). Optionally, each of the biomarkers detectors comprises control indicators for indicating the successfulness of the test, including non-cancerous biomarker detectors and detectors for successful flow of the discharges, such as detectors for the other provided detectors, in the form of secondary antibodies directed to primary antibodies found in the provided detectors.

In some embodiments, the system is provided with a log manager, optionally used for monitoring the changes in discharge biomarker values. In some embodiments, the log manger is manually operated, in the form of a calendar, table, or any fill-out form which could be logged into with a pen. Alternatively or additionally, the log manager is digital, for example in the form of a computer or mobile application. In some embodiments, the system further comprises a sensor, e.g. a camera, optionally, being the camera found in the electronic device used for logging. In some embodiments, the sensor images the results and these images are processed to automatically detect any change in biomarker concentration.

In some embodiments, the system is used periodically at set periods of times, for example once a month, or once every 28 days. In some embodiments, the system further comprises an application for calculating due dates to take the test and optionally also provide a reminder, and/or alarm and/or notification.

FIGS. 9a and b illustrate an example showing a plurality of detectors 4, directed to a cancerous biomarker, each having a different concentration range and threshold, in accordance with some embodiments of the invention. FIG. 9a illustrates a possible baseline, showing only two ranges being indicated, while FIG. 9b illustrates a change in concentration, showing three ranges being indicated.

Reference is now made to FIG. 10, schematically illustrating a personal monitoring system, having at least two cancerous biomarker detectors 4, and one non-cancerous control detector, in accordance with some embodiments of the invention. FIG. 10a illustrates a possible baseline, showing only two ranges being indicated for one biomarker, and three to the second biomarker, while FIG. 10b illustrates a change in concentration, showing four ranges being indicated for each biomarker.

### Example 1

Suggested quantities of marker identifications, along with associated indexes, are depicted in Table 1 below:

| Biomarker | Index | U/ml |
|---|---|---|
| CA125 | A | 0-35 |
| | B | 35-100 |
| | C | 100-250 |
| | D | 250-500 |
| | E | 500-1000 |
| HE-4 | A | 0-35 |
| | B | 35-100 |
| | C | 100-250 |
| | D | 250-500 |
| | E | 500-1000 |

Table 2 below shows an example of a user's fill-out form, calendar, or digital application interface, illustrating a probable outcome when the user's consecutive tests and results indicate diagnosis of a change in relative biomarker quantities:

| Biomarker | Index | 1^{st} test | 2^{nd} test | 3^{rd} test |
|---|---|---|---|---|
| 1 | A | + | + | + |
| [CA125] | B | + | + | + |
| | C | − | − | + |
| | D | − | − | + |
| | E | − | − | − |
| 2 | A | + | + | + |
| [HE-4] | B | + | + | + |
| | C | + | + | + |
| | D | − | − | − |
| | E | − | − | − |
| Results | | 1B/2C | 1B/2C | 1D/2C |

While the invention is susceptible to various modifications and alternative forms, specific embodiments thereof have been shown by way of example in the drawings and the above detailed description. It should be understood, however, that it is not intended to limit the invention to the particular forms disclosed, but on the contrary, the intention is to cover all modifications, equivalents, and alternatives falling within the spirit and scope of the invention as defined by the appended examples.

1.-45. (canceled)

46. A female hygienic device for diagnosing and/or monitoring cancer, comprising:
a. at least one absorption zone for accumulating vaginal discharge; and
b. at least one indication zone comprising at least one agent capable of reacting with a physiological marker and to provide a visual indication of the reaction, wherein the physiologic marker is indicative of a cancerous condition and wherein the physiologic marker is selected from the group consisting of: 22-1-1, leptin, prolactin, osteoponin, inslin-like growth factor II, macrophage inhibitory factor, CD44, soluble CD 54, miRNA 21, miRNA 92, miRNA 93 or any combination thereof.

47. The device of claim 46, wherein said vaginal discharge comprises cervix mucus or fallopian secretions.

48. The device of claim 46, wherein the hygienic device further comprises at least one viscosity-reducing agent.

49. The device of claim 48, wherein the at least one viscosity-reducing agent comprises an agent for targeting an enzymatic activity of mucin.

50. The device of claim 49, wherein the viscosity-reducing agent is selected from the group consisting of water beads, polyacrylamide, superabsorbent polymer, N- acetyl-L-cystein, N-acetylcytamine, Sodium thioglycollate, 2-mercaptoethanol, B-mercaptoethanol, viscosity reducing enzymes, acetic acid, ammonium acetate, (NH4)2SO4, perchloric acid, NaOH, DTT, Urea, SDS, Sodium Chloride and any combination thereof.

51. The device of claim 46, wherein the indication zone comprises a lateral flow test strip.

52. The device of claim 51, wherein the lateral flow strip comprises at least two areas comprising the at least one agent, wherein each of the at least two areas comprises a different amount of the at least one agent.

53. The device of claim 46, the said indication zone further comprises at least one second agent for visually reacting with a second physiologic marker.

54. The device of claim 53, wherein the second physiologic marker is a not indicative of a cancerous condition.

55. The device of claim 54, wherein the second physiologic marker comprises at least one of the group consisting of albumin, actin, tubulin, a secondary antibody and any combination thereof.

56. The device of claim 46, wherein the female hygienic device is a sanitary pad or a tampon.

57. The device of claim 46, wherein the female hygienic device further comprises a barrier film having at least one orifice, the at least one orifice aligned with the at least one absorption zone.

58. The device of claim 46, wherein the agent comprises a DNA probe, an RNA probe or an antibody.

59. The device of claim 46, wherein the at least one indication zone further comprises a detection enhancer, wherein said detection enhancer is selected from the group consisting of gold nanoparticles, gold microparticles, polysteren beads, cellulose nanobeads and any combination thereof.

60. The device of claim 46, wherein the visual indication comprises a color change and/or a color intensity change.

61. The device of claim 46, wherein the visual indication is indicative of a predetermined concentration of the physiological marker.

62. The device of claim 46, further comprising a visual indicator for indicating a predetermined quantity of the vaginal discharge.

63. A method for diagnosing and/or monitoring cancer with a female hygienic device, comprising:
a. absorbing in a hygienic device vaginal discharges of a user;
b. detecting at least one physiologic marker in the vaginal discharge, the physiologic marker being indicative of a cancerous condition and selected from the group consisting of: 22-1-1, leptin, prolactin, osteoponin, inslin-like growth factor II, macrophage inhibitory factor, CD44, soluble CD 54, miRNA 21, miRNA 92, miRNA 93, or any combination thereof; and
c. visually presenting the detection of the at least one physiological marker.

64. The method of claim 63, wherein said vaginal discharge comprises cervix mucus or fallopian secretions.

65. The method of claim 63, wherein said detecting is conducted by allowing a viscosity-reducing agent to contact the vaginal discharge.
